(19) United States
(12) Patent Application Publication
(10) Pub. No.: US 2024/0102991 A1
(43) Pub. Date: Mar. 28, 2024

(54) Synthetic Fluorescent Protein Biosensors and Use Thereof in Drug Screening Methods

(71) Applicant: The Regents of the University of Colorado, a body corporate, Denver, CO (US)

(72) Inventors: Xuedong Liu, Niwot, CO (US); Douglas A. Chapnick, Boulder, CO (US); William Old, Boulder, CO (US); Tristan D. Mcclure-Begley, Falls Church, VA (US); Eric Bunker, Derwood, MD (US)

(21) Appl. No.: 18/386,764

(22) Filed: Nov. 3, 2023

Related U.S. Application Data
(63) Continuation of application No. 16/954,100, filed on Jun. 15, 2020, now abandoned.
(60) Provisional application No. 62/599,551, filed on Dec. 15, 2017.

(51) Int. Cl. G01N 33/50; C12M 1/34; C12Q 1/6825; G01N 21/64; G01N 33/542; G01N 33/554; G01N 33/58

(52) U.S. Cl. CPC G01N 33/5008; C12M 1/3476; C12Q 1/6825; G01N 21/64; G01N 21/6428; G01N 21/6452; G01N 33/542; G01N 33/554; G01N 33/582; B01L 2300/0636; C12Q 2500/00; C12Q 2560/00; C12Q 2563/107; C12Q 2563/179; C12Q 2565/1015; C12Q 2600/136; G01N 2500/04; G01N 2800/52

(57) ABSTRACT

Provided are FRET-based biosensor constructs, and multiplexed platforms or arrays of these biosensor constructs useful for screening candidate drug molecules for efficacy and/or specificity of drug activity. Optionally the biosensor constructs may be located on an inner membrane within a cell or engineered to be located on the cell's surface. The cells or cell lines displaying the biosensors on a cell surface may be arranged as an array of cells for high throughput evaluation of the efficacy and/or specificity of drug candidates, such as a library of candidate drug compounds.

### CROSS-REFERENCE TO RELATED APPLICATION

This application is a continuation of U.S. application Ser. No. 16/954,100, filed Jun. 15, 2020; which is a national stage application under 35 U.S.C. 371 of PCT Application No. PCT/US2018/065673 having an international filing date of Dec. 14, 2018, which designated the United States, which PCT application claimed the benefit of U.S. Application Ser. No. 62/599,551, filed Dec. 15, 2017, both of which are incorporated by reference in their entirety.

### STATEMENT OF FEDERALLY SPONSORED RESEARCH

This invention was made with government support under grant number W911NF-14-2-0019 awarded by the U.S. Army Research Office, and grant number GM113141 awarded by the National Institutes of Health. The government has certain rights in the invention.

### TECHNICAL FIELD

This invention relates to molecular and cellular biology and drug discovery and evaluation. Provided are cell-based or multiplexed platforms for monitoring or assaying the activity, specificity, or off-target effects of a drug that may be a protein, enzyme, or small organic molecule therapeutics.

### BACKGROUND

The extensive ongoing effort to treat or cure diseases relies on the fundamental principle that chemicals can change cellular processes. Drug development is largely the practice of finding a chemical that affects an enzyme, or drug target. As drugs progress through drug development, unintended drug responses referred to as ‘side effects’ frequently emerge. Severe side effects, such as heart attack and stroke, can take years to discover. These side effects arise in part because small molecules bind to and affect not one enzyme, but many enzymes in a cell (termed off-target effects). Finding robust ways to predict off-target effects of drugs is critical for improving the efficiency of drug development since the success of drug development relies on the future side effects just as much as the efficacy.

Advances in understanding mechanisms governing basic cellular functions has allowed researchers to shift focus toward cellular dynamics and create a growing demand for methods that are sensitive and sufficiently quick to track dynamic processes within living cells. In the area of subcellular localization of macromolecular interactions, Förster Resonance Energy Transfer (FRET)-based approaches are particularly useful.

Considering the important role of unwanted, off-target effects of candidate therapeutic compounds and the advances in FRET-based screening technologies, there is a desire for sensitive and cost-effective high throughput assays to evaluate the efficacy and specificity of drugs.

### SUMMARY

This disclosure provides methods of producing and utilizing ensembles of synthetic biosensor genes within cells to screen chemical libraries, allowing comparisons between drugs that can inform a high risk for future side effects, as well as find new uses for non-toxic drugs. Production of biosensor gene variant libraries is accomplished by insertion of non-coding or protein-coding DNA fragments into a variable region of a modular biosensor backbone plasmid DNA, resulting in one biosensor variant for each DNA fragment. These biosensor genes are inserted into mammalian cells and the biosensor-containing cells are exposed to a panel of biochemical pathway specific drugs in parallel simultaneous microscopy experiments. Pathway specific biosensors are identified based on their ability to respond to few and not all biochemical pathway specific drugs, where the biosensor translates activation or repression of a biochemical pathway into dynamically changing ratiometric fluorescence intensity between two fluorescent domains in each biosensor, which can be measured quickly and efficiently with a fluorescence detector. The ratiometric fluorescence intensity between two fluorescent domains in each biosensor can be the ratio of either direct fluorescence from each of the two fluorescent domains in each biosensor, or the forster resonance energy transfer ratio (FRET ratio) between each of the two fluorescent domains in each biosensor. Isolated pathway specific biosensors are used in subsequent drug screening efforts to compare effects of candidate drugs on multiple biochemical pathways in parallel simultaneous microscopy experiments.

Thus, this disclosure provides FRET-based protein biosensor constructs that indicate efficacy and specificity of candidate drugs through fluorescence. This disclosure also provides a genetically encoded FRET-based biosensor construct that overcomes the limitations of the currently available biosensors and makes investigation of efficacy and specificity of candidate drugs on membranes of living cells possible.

In one aspect, this disclosure provides methods of conducting dynamic single-molecule fluorescence studies on a membrane protein to evaluate and compare efficacy and specificity of candidate drugs on membrane proteins. These methods generally involve expressing a FRET-based protein biosensor construct of this disclosure on the membrane of a living cell or on the membrane of intracellular organelles within a living cell and exposing the cell to at least one candidate drug and measuring the change in FRET activity or ratiometric fluorescence in response to the drug, which FRET activity or ratiometric fluorescence is indicative of one or more of efficacy and specificity of the candidate drug.

Thus, this disclosure provides a protein biosensor construct comprising a FRET donor fluorescent protein and a FRET acceptor fluorescent protein separated by flexible linkers, wherein the linker between the donor and acceptor proteins comprises a sensing domain and the entire biosensor is anchored in a plasma membrane of a living cell in cell culture. In these biosensor constructs, the sensing domain may comprise a peptide, protein, or nucleic acid that will interact with molecules, such as drug molecules, or molecules in or on or around a cell, resulting in a detectable change in florescence from the FRET donor and acceptor pair within the biosensor construct. The detectable change in florescence from the FRET donor and acceptor pair may result from changes in the physical distance between the FRET donor and acceptor in the biosensor construct and/or from changes in the position of the FRET donor and acceptor in three-dimensional space in the biosensor construct.

Using nucleic acid fragments within the sensing domain in these biosensor constructs allows for generation of multiple biosensor variants, each with the ability to physically interact with endogenous proteins in the biosensor-expressing cell. Dynamic FRET activity or ratiometric fluorescence from each biosensor results from distortion of the three-dimensional shape of the biosensor construct following physical contact between intracellular molecules in the biosensor-expressing cell and the sensing domain. The physical contact may include proteolytic cleavage of a peptide bond at one or many sites by proteolytic enzymes within the biosensor expressing cell.

An exemplary biosensor construct of this disclosure comprises, covalently linked from N-terminus to C-terminus: 1) a FRET acceptor or donor; 2) a proteolytic cleavage site; 3) a ligand; 4) a FRET acceptor or donor that completes a FRET energy transfer pair with the first position; 5) a ligand binding domain; and, 6) a protease specific for the proteolytic cleavage site. In this exemplary biosensor, the FRET acceptors or donors in the first and fourth positions may comprise a fluorescent resonance energy transfer (FRET) pair, such that cleavage of the proteolytic cleavage site and/or inhibition of binding between the ligand and the ligand-binding domain sites results in a change in the distance or orientation between the FRET donor and FRET acceptor.

In these biosensors, one or more flexible peptide linkers may be used to link at least two of the construct components such that the ligand and the ligand-binding domain may freely interact within each biosensor molecule.

In embodiments, these biosensors may be anchored in a plasma membrane of a living cell in cell culture. Anchoring the biosensor in a membrane may be achieved using a hydrophobic protein domain or a domain that can be modified with lipids by lipid conjugation enzymes.

Another exemplary biosensor construct of this disclosure comprises, covalently linked from N-terminus to C-terminus: 1) a FRET acceptor or donor; 2) a sensing domain comprising a peptide or protein fragment encoded by DNA fragments resulting from enzymatic processing of double stranded coding and non-coding mammalian DNA or complimentary DNA (cDNA); and 3) a FRET acceptor or donor that completes a FRET energy transfer pair with the first position. In this second exemplary biosensor, the FRET acceptors or donors in the first and third positions may comprise a fluorescent resonance energy transfer (FRET) pair, such that binding of proteins to the sensing domain results in a change in the distance or orientation between the FRET donor and FRET acceptor groups, resulting in a detectable change in dynamic FRET activity or ratiometric fluorescence from each biosensor.

This disclosure also provides an isolated, recombinant, synthetic, or chimeric, nucleic acid encoding any one of these biosensor constructs, operatively linked to an inducible promoter or a constitutive promoter. These nucleic acids encoding these biosensor constructs may be positioned within a vector, expression cassette, cosmid, or plasmid, which may be infected or transfected into a living cell.

This disclosure also provides a biosensor array comprising a plurality of the biosensor constructs of this disclosure and a substrate having a surface, wherein each of the biosensors is anchored to the substrate surface at an addressable site in the array. The array of biosensors may comprise at least two biosensors comprising different proteolytic cleavage sites and/or different ligands in the biosensor constructs. In these arrays, the plurality of biosensors may be anchored to the substrate by displaying each biosensor on the surface of a living cell in cell culture at each addressable site in the array.

Thus, this disclosure also provides an array of cells in cell culture wherein each cell displays a biosensor on a surface of each cell in the array. These array of cells may contain at least two biosensors, each comprising different sensing domains (such as proteolytic cleavage sites) in the biosensor constructs. These array of cells may contain at least two biosensor constructs of this disclosure, each comprising different ligands in the biosensor constructs. These array of cells may contain at least two biosensor constructs, each comprising different sensing domains in the biosensor constructs.

This disclosure also provides a kit comprising at least one kit component selected from: a biosensor construct of this disclosure; an isolated, recombinant, synthetic, or chimeric, nucleic acid encoding a biosensor of this disclosure; a vector, expression cassette, cosmid, or plasmid comprising a nucleic acid of this disclosure; a biosensor array of this disclosure; and/or an array of cells of this disclosure. These kits include instructions that explain at least one of: how to conduct a candidate drug evaluation assay using the kit components, how to detect fluorescence from the kit components, and/or how to correlate fluorescence or ratiometric fluorescence from a biosensor to candidate drug activity or specificity.

This disclosure also provides methods of evaluating drug activity by detecting whether the drug has activity in modulating ligand binding, proteolytic activity, or endogenous protein binding by contacting at least one biosensor of this disclosure with the drug, illuminating the sample, detecting fluorescence resulting from the FRET energy transfer pair; and determining the drug activity in the sample by comparing the fluorescence or ratiometric fluorescence resulting from drug contact with the biosensor construct with a reference correlation of fluorescence or ratiometric fluorescence and known or standard drug activity.

In these methods, drug specificity may be evaluated by contacting the drug with at least two biosensors, each comprising different proteolytic cleavage sites, and comparing the fluorescence resulting from drug contact with the different biosensor constructs to determine specificity of the drug in modulating the proteolytic cleavage of each biosensor. Similarly, drug specificity may also be evaluated in these methods by contacting the drug with at least two biosensors, each comprising different ligands, and comparing the fluorescence resulting from drug contact with the different biosensor constructs to determine specificity of the drug in modulating the ligand-ligand binding domain interaction of each biosensor, or interactions between endogenous cellular proteins and the sensing domain of the biosensor constructs. In these methods, the at least one biosensor may be composed as an array of biosensors, wherein the array of biosensors includes one or more different proteolytic cleavage sites, and/or ligands, and/or sensing domains. In these methods, the array of biosensors may be displayed on a surface of living cells arranged in an array of cells in cell culture. In these methods, the array of biosensors may be displayed on a surface of subcellular organelles within living cells arranged in an array of cells in cell culture. In these methods, the drug may be contacted with the at least one biosensor as part of a composition comprising a library of candidate drugs such that the fluorescence detected from the array of biosensors provides a comparison of drugs present in the library of candidate drugs.

This Summary is neither intended nor should it be construed as being representative of the full extent and scope of the present invention. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached figures and the Detailed Description and no limitation as to the scope of the present disclosure is intended by either the inclusion or non-inclusion of elements, components, etc. in this Summary. Additional aspects of the present disclosure will become more readily apparent from the Detailed Description, particularly when taken together with the figures.

### BRIEF DESCRIPTION OF FIGURES

FIG. 1 depicts one example of a FRET-based biosensor construct of this disclosure.

FIG. 2 depicts the molecular events that transition the FRET-based biosensor construct of FIG. 1 from high FRET to low FRET activity.

FIG. 3 depicts the construction and display of a FRET-based biosensor construct of this disclosure on a cell membrane.

FIG. 4 depicts a method of high throughput drug screening using an array of FRET-based biosensor constructs of this disclosure located on an array of cell membranes using fluorescence detection and data display.

FIG. 5 depicts cellular array drug testing data collected in an exemplary method of high throughput drug screening using an array of FRET-based biosensor constructs of this disclosure.

FIG. 6 is a diagram of a modular biosensor backbone design of this disclosure.

FIG. 7 is shows fluorescence from biosensor constructs of this disclosure located on intracellular cell membranes (left) and external cell membranes (right).

FIG. 8 depicts the molecular interactions between molecules and the biosensor constructs of this disclosure, leading to detectable changes in FRET fluorescence.

FIG. 9 shows the results of time-dependent (2 hr) biosensor profiling of single agent drug response.

FIG. 10 shows the results of time-dependent (3 hr) biosensor profiling of combinatorial drug responses.

### DETAILED DESCRIPTION

This disclosure provides FRET-based biosensor constructs and genetically encoded biosensors that are useful in high throughput assays of candidate drug efficacy and specificity that enhance the speed, cost effectiveness, and accuracy of drug discovery.

The following definitions are provided to facilitate an understanding of the present disclosure:

The singular forms “a,” “an,” and “the” include plural referents unless the context clearly dictates otherwise.

As used herein, the term “small molecule” refers to a substance or compound that has a relatively low molecular weight (e.g., less than 4,000 atomic mass units (a.m.u.), particularly less than 2,000 a.m.u.). Typically, small molecules are organic, but are not proteins, polypeptides, or nucleic acids, though they may be amino acids or dipeptides.

The term “isolated” may refer to a compound or complex that has been sufficiently separated from other compounds with which it would naturally be associated. “Isolated” is not meant to exclude artificial or synthetic mixtures from other compounds or materials, or the presence of impurities that do not interfere with fundamental activity or ensuing assays, and that may be present, for example, due to incomplete purification, or the addition of stabilizers.

“Nucleic acid” or a “nucleic acid molecule” as used herein refers to any DNA or RNA molecule, either single or double stranded and, if single stranded, the molecule of its complementary sequence in either linear or circular form.

“Antisense molecule” refers to a nucleic acid molecule that hybridizes to all or a portion of a target gene or all or a portion of an mRNA encoded by a target gene. Such antisense molecules are typically between 15 and 30 nucleotides in length and often span the translational start site of mRNA molecules.

The term “treat” as used herein refers to any type of treatment that imparts a benefit to a patient afflicted with a disease or disorder or infection, including improvement in the condition of the patient (e.g., in one or more symptoms), delay in the progression of the condition, etc.

As used herein, the term “prevent” refers to the prophylactic treatment of a subject who is at risk of developing a condition (e.g., bacterial infection) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof. For example, “therapeutically effective amount” may refer to an amount sufficient to modulate stress and/or stress response in a subject.

As used herein, the term “subject” refers to an animal, particularly a mammal, particularly a human.

As used herein, the phrases “fluorescence resonance energy transfer” and “FRET” refer to the energy transfer from an excited fluorescent group to, at least partially, a quenching/fluorescent group. The quenching/fluorescent group may radiate the absorbed light as light of a different wavelength or dissipate it as heat. FRET depends on 1) an overlap between the emission spectrum of the fluorescent group and the absorption spectrum of the quenching/fluorescent group and 2) the distance between the quenching/fluorescent group and the fluorescent group. Above a certain critical distance, the quenching/fluorescent group is unable to absorb the light emitted by the fluorescent group.

As used herein, the term “fluorescent group” (sometimes referred to as a fluorophore or FRET donor) refers to a molecule that, when excited with light having a selected wavelength, emits light of a different wavelength. A quenching/fluorescent group refers to a group that can absorb at least partly the energy/light emitted by a fluorescent group. Depending on the identity of the quenching/fluorescent group, the fluorescence emission can undergo a number of alterations, including, but not limited to, attenuation, complete quenching, a shift in wavelength, a shift in polarity, and a change in fluorescence lifetime. For example, the quenching/fluorescent group may radiate the absorbed light as light of a different wavelength.

As used herein, the term “energy transfer pair” refers to any two molecules that participate in fluorescence resonance energy transfer. The energy transfer pair is typically two molecules that participate in fluorescence resonance energy transfer. The energy transfer pair may comprise a first energy transfer pair member and a second energy transfer pair member. Typically, the first energy transfer pair member is different than the second energy transfer pair member.

As used herein, the term “array” refers to an ordered arrangement of array elements. The array elements are arranged so that there are at least one or more different array elements. The array elements may be contained within/on a solid support or on or in living cells, which cells are arranged as an array of elements. For example, an array of cells may be contained in a microtiter plate.

A “solid support” refers to any solid surface including, without limitation, any chip (for example, silica-based, glass, or gold chip), glass slide, membrane, plate, bead, solid particle (for example, agarose, sepharose, polystyrene or magnetic bead), column (or column material), test tube, or microtiter dish/plate.

The term “operably linked” refers to a juxtaposition/linkage wherein the components so described are in a relationship permitting them to function in their intended manner.

“Single-molecule fluorescence resonance energy transfer” (or “smFRET”) is the application of FRET techniques to study a single molecule with at least two fluorescent labels, or the interaction of at least two molecules, each with a label. Fluorescence Resonance Energy Transfer (FRET) is a non-radiative pathway by which a molecule in an electronic excited state may relax back to the more stable ground state. The transfer of energy occurs through space via dipole-dipole interaction: energy from the excited-state molecule (the donor fluorophore) may transfer to a neighboring molecule (the acceptor fluorophore) given significant degree of spectral overlap between donor emission and acceptor absorption, properly oriented dipole moments of the interacting dye molecules, and the appropriate distance between the two fluorophores. In smFRET, the donor and receptor fluorophores are either on the same molecule, or are on different molecules that interact, bringing the two fluorophores into proximity The detection of FRET at the single-molecule scale enables the direct measurement of conformational events on biologically-relevant time scales. At least two fluorophores are required.

A “fluorophore” is a component of a molecule which causes a molecule to be fluorescent. It is a functional group in a molecule which will absorb energy of a specific wavelength and re-emit energy at a specific wavelength. The amount and wavelength of the emitted energy depend on both the fluorophore and the chemical environment of the fluorophore. Fluorescein isothiocyanate (FITC), a reactive derivative of fluorescein, has been one of the most common fluorophores chemically attached to other, non-fluorescent molecules to create new fluorescent molecules for a variety of applications. Other common fluorophores are derivatives of rhodamine (TRITC), coumarin, pyrene, and cyanine. Newer generations of fluorophores such as maleimide derivative dyes, CF dyes, the FluoProbes dyes, the DyLight Fluors, the Oyester dyes, the Atto dyes, the HiLyte Fluors, and the Alexa Fluors are believed to perform better (more photostable, brighter, and/or less pH-sensitive) than other standard dyes of comparable excitation and emission. A molecule containing a fluorophore may also be referred to herein as a “dye”. A skilled artisan will recognize that many fluorophore molecules are suitable for FRET constructs and FRET analysis. Fluorescent proteins may be used as fluorophores. Representative donor fluorescent moieties that can be used with various acceptor fluorescent moieties in FRET technology include cyan fluorescent proteins (CFP), fluorescein, Lucifer Yellow, B-phycoerythrin, 9-acridineisothiocyanate, Lucifer Yellow VS, 4-acetamido-4′-isothio-cyana tostilbene-2,2′-disulfonic acid, 7 diethylamino-3-(4′-isothiocyanatophenyl)-4-methylcoumarin, succinimdyl 1-pyrenebutyrate, and 4-acetamido-4′-isothiocyanatostilbene-2-,2′-disulfonic acid derivatives. Representative acceptor fluorescent moieties, depending upon the donor fluorescent moiety used, include yellow fluorescent proteins (YFP), LC-Red 640, LC-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

Examples of fluorescence proteins or labels that may be used in the FRET constructs of this disclosure include fluorescein, 6-FAM™ (Applied Biosystems, Carlsbad, Calif.), TET™ (Applied Biosystems, Carlsbad, Calif.), VIC™ (Applied Biosystems, Carlsbad, Calif.), MAX, HEX™ (Applied Biosystems, Carlsbad, Calif.), TYE™ (ThermoFisher Scientific, Waltham, Mass.), TYE665, TYE705, TEX, JOE, Cy™ (Amersham Biosciences, Piscataway, N.J.) dyes (Cy2, Cy3, Cy3B, Cy3.5, Cy5, Cy5.5, Cy7), Texas Red® (Molecular Probes, Inc., Eugene, Oreg.), Texas Red-X, AlexaFluor® (Molecular Probes, Inc., Eugene, Oreg.) dyes (AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750), DyLight™ (ThermoFisher Scientific, Waltham, Mass.) dyes (DyLight 350, DyLight 405, DyLight 488, DyLight 549, DyLight 594, DyLight 633, DyLight 649, DyLight 755), ATTO™ (ATTO-TEC GmbH, Siegen, Germany) dyes (ATTO 390, ATTO 425, ATTO 465, ATTO 488, ATTO 495, ATTO 520, ATTO 532, ATTO 550, ATTO 565, ATTO Rho101, ATTO 590, ATTO 594, ATTO 610, ATTO 620, ATTO 633, ATTO 635, ATTO 637, ATTO 647, ATTO 647N, ATTO 655, ATTO 665, ATTO 680, ATTO 700, ATTO 725, ATTO 740), BODIPY® (Molecular Probes, Inc., Eugene, Oreg.) dyes (BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665), HiLyte Fluor™ (AnaSpec, Fremont, Calif.) dyes (HiLyte Fluor 488, HiLyte Fluor 555, HiLyte Fluor 594, HiLyte Fluor 647, HiLyte Fluor 680, HiLyte Fluor 750), AMCA, AMCA-S, Cascade® Blue (Molecular Probes, Inc., Eugene, Oreg.), Cascade Yellow, Coumarin, Hydroxycoumarin, Rhodamine Green™-X (Molecular Probes, Inc., Eugene, Oreg.), Rhodamine Red™-X (Molecular Probes, Inc., Eugene, Oreg.), Rhodamine 6G, TMR, TAMRA™ (Applied Biosystems, Carlsbad, Calif.), 5-TAMRA, ROX™ (Applied Biosystems, Carlsbad, Calif.), Oregon Green® (Life Technologies, Grand Island, N.Y.), Oregon Green 500, IRDye® 700 (Li-Cor Biosciences, Lincoln, Nebr.), IRDye 800, WellRED D2, WellRED D3, WellRED D4, and Lightcycler® 640 (Roche Diagnostics GmbH, Mannheim, Germany). Suitable acceptors include Black Hole Quencher®-1 (Biosearch Technologies, Novato, Calif.), BHQ-2, Dabcyl, Iowa Black® FQ (Integrated DNA Technologies, Coralville, Iowa), IowaBlack RQ, QXL™ (AnaSpec, Fremont, Calif.), QSY 7, QSY 9, QSY 21, QSY 35, and IRDye QC.

### Biosensors

The inventors have designed a novel, versatile backbone design to generate a large array of biosensors, which can each measure specific biochemical events within biochemical pathways in, or on the surface of, a cell. This design overcomes a major problem with existing biosensors that lack a tunable dynamic range suitable to report changes in biochemical events that occur during normal and stress- or stimuli-induced cellular processes. The novel design allows for each molecule of a biosensor in a cell to exist in either an intact or cleaved state, depending on whether the intramolecular ligand binds the ligand binding domain. Upon binding between these two components of the biosensor, a protease domain cleaves a proteolytic cleavage site, releasing the terminal FRET fluorophore, and decreasing measured FRET within the sensor.

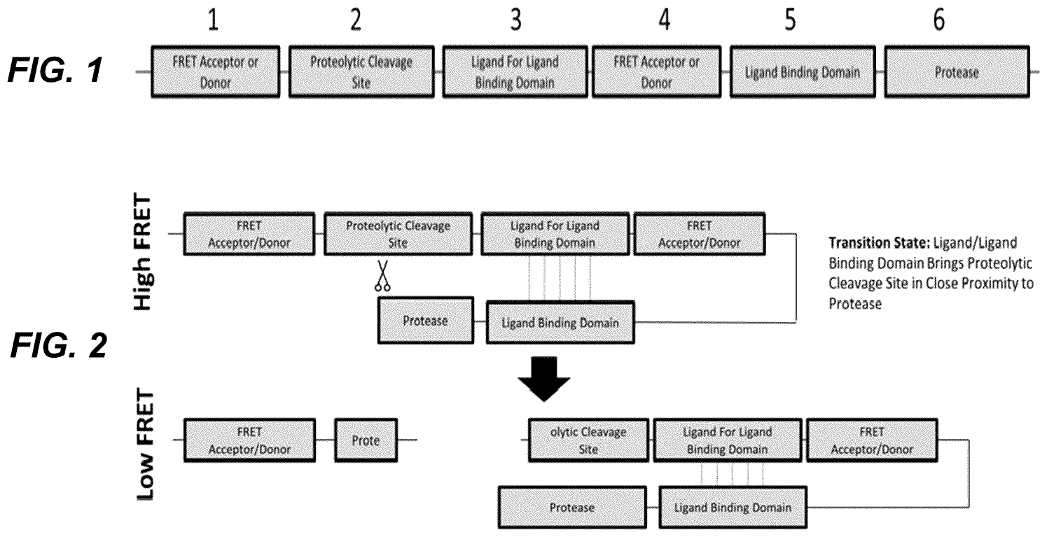

Referring to FIG. 1, an exemplary biosensor of this disclosure is diagramed comprising six specific domains or positions that are covalently linked to form the biosensor construct. As shown in FIG. 1, positions 1 and 4 form a FRET donor/acceptor fluorophore energy transfer pair, where donor and acceptor are exchangeable at these positions, wherein if the donor is placed at position 1 then acceptor is placed at position 3, or visa-versa. Positions 2 and 6 are ideal proteolytic substrate domain and paired protease, respectively. By altering positions 3 and/or 5, variations in biosensor specificity can be generated, wherein cellular biomolecules interact with, or modify, the ligand domain, resulting in either activation or repression of the biosensor intramolecular interaction between positions 3 and 5, thereby enhancing or repressing the proteolytic activity of position 6 on position 2, and enhancing or repressing FRET energy exchange between positions 1 and 4, resulting in detectable changes in fluorescence or ratiometric fluorescence from the biosensor construct. As shown in FIG. 1, the diagrammed biosensor exists in a high FRET constrained confirmation, where the FRET donor and acceptor are covalently linked in close proximity.

Thus, one biosensor construct of this disclosure comprises (from N-terminus to C-terminus):
a FRET acceptor or donor;
a proteolytic cleavage site;
a ligand;
a FRET acceptor or donor that completes the FRET energy transfer pair with the first position;
a ligand binding domain; and,
a protease specific for the proteolytic cleavage site in the second position.

These elements in the biosensor are optionally linked through one or more flexible linkers positioned between each element, such that the linker allows interaction between these elements within the biosensor construct. These flexible linkers are typically peptide(s) that may be positioned to separate the donor and acceptor fluorophores in the FRET biosensor construct by a distance of not more than 10 nm, to allow detectable fluorescence from the intact biosensor construct.

The linker peptide of these FRET constructs may comprise at least about six amino acid residues and an amino acid sequence selected from the group consisting of SEQ ID NOs: 1-4. These linker peptides may comprise at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22 amino acid residues, or at least about 25 amino acid residues, or more than about 25 amino acid residues. These linker peptides may also comprise longer amino acid sequences, including sequences of at least about 30 amino acid residues, or at least about 40 amino acid residues, or at least about 45 amino acid residues, or at least about 50 amino acid residues, or at least about 55 amino acid residues, or at least about 65 amino acid residues.

The linker sensing domain containing a nucleic acid may include any nucleic acid (DNA and/or RNA) and may comprise at least about six nucleic acid residues. These sensing domain nucleotides may comprise at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22 nucleic acid residues, or at least 25 nucleic acid residues, or more than 25 nucleic acid residues. These sensing domain nucleic acids may also comprise longer amino acid sequences, including sequences of at least about 30 nucleic acid residues, or at least about 40 nucleic acid residues, or at least about 45 nucleic acid residues, or at least about 50 nucleic acid residues, or at least about 55 nucleic acid residues, or at least about 65 nucleic acid residues.

The FRET acceptor/donor energy transfer pair (in the first and fourth positions of the biosensor construct depicted in FIG. 1) may comprise one or more amino acids in these two positions that have been labeled (i.e., covalently linked directly or through a linker as described above) with a donor or acceptor fluorescent moiety.

Referring to FIG. 2, this biosensor construct senses activity and specificity of candidate drug molecules when the intact biosensor maintains sufficiently close proximity between the FRET acceptor and donor energy transfer pair to fluoresce. The intra-molecular interaction between ligand and ligand-binding domain is coupled to a proteolytic event that cleaves the sensor and causes dramatic reduction in FRET between FRET donor and FRET acceptor fluorophores. The ligand is bound by the ligand binding domain, thereby bringing the protease into proximity of the proteolytic cleavage site. Proteolytic cleavage of the cleavage site splits the biosensor, separating the FRET acceptor donor energy transfer pair, abolishing the fluoresce. In this way, any candidate drug molecule that interacts with (e.g., inhibits or competes with) the ligand-ligand binding domain interaction will reduce or eliminate fluoresce from the FRET acceptor donor energy transfer pair. This change in fluorescence may be detected as a response to the candidate drug molecule's effect on the ligand-ligand binding domain interaction. Using an array of such biosensors, each comprising different ligand-ligand binding domains, a candidate drug molecule's effects may be evaluated across a wide range of drug-biomolecule interactions, as detected by changes in fluoresce from the FRET acceptor donor energy transfer pair, thereby providing a rapid and efficient measurement of the drug's efficacy and specificity, or lack thereof.

These biosensor constructs may further comprise a transmembrane domain or moiety that attaches the FRET biosensor construct to the external surface of a cell, or on the outer surface of a membrane, a liposome or an exosome. Such transmembrane domain may locate and/or anchor the FRET biosensor construct to a cellular membrane (an intracellular membrane, such as the membrane of a cellular organelle, or the external cell membrane) in a particular orientation or location to enhance the testing of candidate drug molecule's interaction with membrane proteins including the FRET biosensor construct, and potentially other membrane proteins.

This disclosure also provides isolated, recombinant, synthetic, or chimeric polynucleotide molecules encoding a FRET biosensor construct of this disclosure, optionally operatively linked to a promoter, that may be an inducible promoter or a constitutive promoter. This disclosure also provides a cell comprising one or more of these isolated polynucleotide molecules. For example, the cell may be a CHO cell, an E. coli cell, a yeast cell, or a human cell in cell culture, including, for example, a human keratinocyte cell in cell culture located in an array of wells in a microtiter plate.

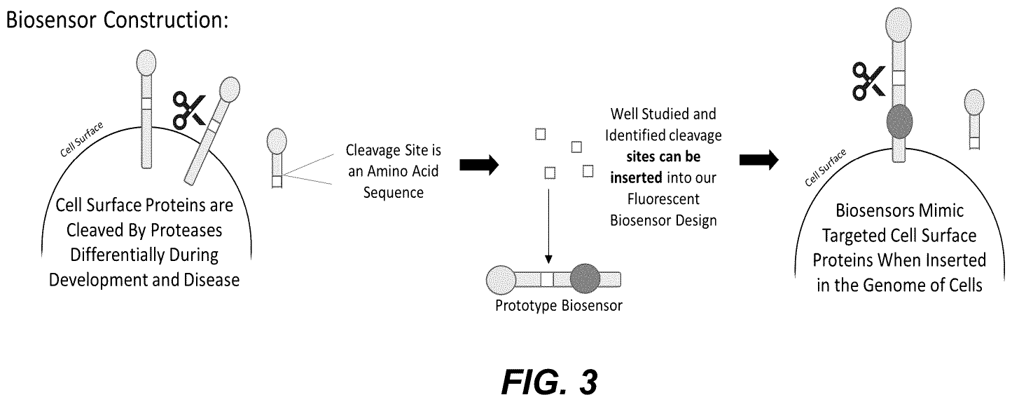

Referring to FIG. 3, living cells may be transfected with these nucleic acids to express the FRET biosensor constructs described above. The FRET biosensor construct proteins may then locate on a surface of the cell. Contacting the cell with candidate drug molecule(s) may modulate the fluorescence from the FRET energy pair, as described above, which modulation may be detected on the surface of the cell. Cells expressing the FRET biosensor constructs on their surface may be arranged in an array to facilitate high throughput screening of candidate drug molecules to evaluate the activity and/or specificity of the drug molecules effect on the ligand-ligand binding domain interaction on the surface of the cells.

In a related aspect, this disclosure also provides methods of making a library of biosensor constructs. In these methods, a library of biosensor constructs is produced by generating at least two polynucleotides comprising different nucleic acid sequences. These different polynucleotides are inserted into separate biosensor constructs such that each biosensor construct contains (from N-terminus to C-terminus) 1) a FRET acceptor or donor; a sensing domain comprising one of the polynucleotides; and 3) a FRET acceptor or donor that completes a FRET energy transfer pair with the first position. These separate biosensor constructs, each containing a different polynucleotide in the sensing domain, together form a library of biosensor constructs comprising sensing domains comprising different nucleic acid sequences. In these methods, the library of biosensor constructs may include polynucleotides with two or more distinct nucleic acid sequences. In these methods, the size of the library may vary. For example, the library may include 2 or more, 3 or more, 4 or more, 5 or more, 10 or more, 25 or more, 100 or more, 500 or more, 1000 or more, 10000 or more, 50000 or more, 100000 or more, 500000 or more, or 1 million or more distinct nucleic acid sequences forming the polynucleotides in the sensing domain of separate biosensor constructs in the library of biosensor constructs. In certain aspects, the library includes two or more, but 1 million or less, 500000 or less, 100000 or less, 50000 or less, 10000 or less, 1000 or less, 500 or less, 100 or less, 25 or less, 10 or less, 5, 4, or 3 distinct nucleic acid sequences forming the polynucleotides in the sensing domain of separate biosensor constructs in the library of biosensor constructs. Known methods of making a library of polynucleotides comprising different nucleic acid sequences may be used to prepare the polynucleotides inserted into the biosensor constructs of this disclosure to form these libraries of biosensor constructs. Such methods may include producing a plurality of distinct nucleic acid sequences using PCR-based methods, and/or methods that include combining a plurality of distinct nucleic acids sequences with a one or more distinct nucleases in a manner sufficient to produce the library of distinct nucleic acid sequences, and/or by transcribing distinct nucleic acid sequences from plasmids encoding such sequences, and/or by solid phase synthesis (e.g., as in standard oligonucleotide synthesis).

The biosensor libraries of this disclosure may be prepared or present in any suitable environment. For example, the biosensor construct library may be present in a composition, such as an aqueous solution or suspension in a reaction tube or a well of a multiwell reaction plate. Other suitable environments include, for example, a microfluidic chip (e.g., a “lab-on-a-chip device”), or an array of cells, as described in detail below.

### Gene Deliver of Biosensors

This disclosure also provides cell-based methods, a cell-based platform or system, or a multiplexed platform or system, for monitoring or assaying the activity and/or specificity of a candidate drug molecule (which may be an enzyme or a protease drug) on a cell, a population of cells, or a culture of cells, or on the outer surface of a membrane, a liposome or an exosome, by transferring, transfecting, transducing, infecting or implanting one or more nucleic acids encoding a FRET-based biosensor construct of this disclosure into the cell or cells or onto the liposome or exosome.

Any protocol, method or means of transferring, transfecting, transducing, infecting, or implanting nucleic acids into cells can be used to practice embodiments as provided herein. For example, in practicing these methods, any known construct or expression vehicle, e.g., expression cassette, plasmid, vector, virus (e.g., retroviral or lentiviral expression vectors or recombinant viruses), and the like, comprising a nucleic acid encoding a readable or detectable moiety, e.g., for use as ex vivo or in vitro gene therapy vehicles, or for expression of a readable or detectable moiety in a target cell, tissue or organ to practice the high throughput drug screening methods provided herein, e.g., for research, diagnosis, therapy, or drug discovery.

As described above, polynucleotides encoding the FRET-based biosensors of this disclosure may include expression cassettes comprising the biosensor-encoding nucleic acid operably linked to a transcriptional regulatory element, e.g., a promoter.

An expression vehicle used to transfect cells, as provided herein, is designed to deliver a biosensor construct-encoding sequence to a tissue or cell. Expression vehicles, e.g., vectors, can be non-viral or viral vectors or combinations thereof. Any viral vector or viral delivery system known in the art may be used, e.g., adenoviral vectors, adeno-associated viral (AAV) vectors, herpes viral vectors (e.g., herpes simplex virus (HSV)-based vectors), retroviral vectors, or lentiviral vectors. The expression vehicle, e.g., a vector or a virus, may be capable of accommodating a full-length gene or a message, e.g., a cDNA, and delivering the nucleotide sequence encoding FRET biosensor construct in vitro, ex vivo and/or in vivo.

Vectors, recombinant viruses, and other expression systems used to transfect the polynucleotides encoding the FRET biosensor constructs can infect, transfect, transiently or permanently transduce a cell. In one aspect, a vector encoding a FRET based biosensor construct of this disclosure may be a naked nucleic acid, or a nucleic acid complexed with protein or lipid.

Vectors used to make or practice embodiments as provided herein can be chosen from any number of suitable vectors known to those skilled in the art, including any plasmid, which are commercially available, publicly available on an unrestricted basis, or can be constructed from available plasmids in accord with published procedures, cosmids, YACs (Yeast Artificial Chromosomes), megaYACS, BACs (Bacterial Artificial Chromosomes), PACs (P1 Artificial Chromosome), MACs (Mammalian Artificial Chromosomes), a whole chromosome, or a small whole genome. The vector also can be in the form of a plasmid, a viral particle, or a phage. Other vectors include chromosomal, non-chromosomal and synthetic DNA sequences, derivatives of SV40, bacterial plasmids, phage DNA, baculovirus, yeast plasmids, vectors derived from combinations of plasmids and phage DNA, viral DNA such as vaccinia, adenovirus, fowl pox virus, and pseudorabies. Bacterial vectors which can be used include commercially available plasmids comprising genetic elements of known cloning vectors.

Thus, this disclosure also provides a recombinant microorganism or cell culture useful in the high throughput drug screening assays provided herein comprising an expression vector including both (or either) extra-chromosomal circular and/or linear nucleic acid (DNA or RNA) that has been incorporated into the host chromosome(s). Where a vector is being maintained by a host cell, the vector may either be stably replicated by the cells during mitosis as an autonomous structure, or is incorporated within the host's genome.

### Biosensor Arrays

A plurality of FRET biosensor constructs of this disclosure may be immobilized, directly or indirectly, to a support or substrate or cell surface to form an array of biosensors. Supports or substrates can take a variety of forms such as polymers, glass, metal and those with coatings thereon. Cells may be eukaryotic cells in cell culture arrayed within wells of a microtiter plate, such as a 96-well microplate. Arrays are ordered arrangements of elements, allowing them to be displayed and examined in parallel. Arrays of immobilized biosensors can be used to detect the effects and the specificity of chemicals on the biosensors comprising different ligand-ligand binding domain combinations. Certain array formats are referred to as “biochips.” Biochips may include a plurality of locations configured so that each location is spatially addressable. Typically, the biosensor array format is configured in a row and column format with regular spacing between locations, wherein each location has machine-readable (e.g., computer-readable) information to identify the location of biosensor within the array.

The affinity and specificity of a candidate drug compound interacting with (e.g., binding to, or interfering with) a ligand or ligand-binding domain is determined using the biosensor microarray described above. Biosensor proteins are immobilized or displayed on a cell surface in an array, and fluorescence from FRET is measured using an array reader. Binding specificity is evaluated first from the binding profile of biosensors to a series of candidate drugs. Fluorescence events are sensed with the binding of the ligand and ligand-binding domain, which brings the proteolytic cleavage site and the proteolytic domain into proximity causing cleavage at the proteolytic cleavage site and separation of the FRET energy pair domains. The fluorescence sensing between different biosensors in the array and between different candidate drugs is correlated with a change in the FRET signal. Thus, absence of activity or specificity generates a specific FRET signal in terms of the wavelength and amplitude of the emission, and the presence of specific domain binding or activity generates a modulated FRET signal emission in terms of either the wavelength, or amplitude, or both.

Fluorescence signals sufficient to be detected within an array of cells expressing the biosensor constructs using a microplate spectrofluorometer may be generated by as low as 300 nM of the FRET biosensor constructs of this disclosure. The fluorescence signal change can be traced in real time to reflect the biochemical effects of the drug. Real time monitoring measures signal changes as a reaction progresses, and allows both rapid data collection and yields information regarding reaction kinetics under various conditions. FRET ratio changes and degrees of binding or inhibition may be correlated, for example for a certain spectrofluorometer using a method such as HPLC assay in order to correlate the unit of kinetic constant from the FRET ratio to candidate drug concentration.

### High Throughput Drug Screening

As described above, methods of the present invention are amenable to high-throughput screening (HTS) formats because the use of fluorescence and FRET-based assays is a standard platform known in the art for HTS. In these HTS assays, the modulation of ligand binding, proteolytic enzyme activity, or biosensor binding to endogenous proteins is performed within an array of biosensors (e.g., in a multi-well microassay plate comprising living cells displaying at least one biosensor of this disclosure on the surface of each cell). Such microarrays allow for researchers to quickly and cost-effectively screen numerous candidate drugs (e.g., a library of drugs, such as several different inhibitor compounds) at the same time.

Thus, this disclosure further provides a method for high throughput screening for compounds that modulate (e.g., inhibit) proteolytic enzyme activity or compounds that modulate (e.g., inhibit) the ligand-ligand binding domain interaction using the above described arrays of biosensors immobilized on a surface or expressed on a cell surface. Because of its high sensitivity, rapid readout, and ease of use, in vitro arrays of this disclosure are also suitable for screening candidate drug libraries. Specifically, a FRET biosensor construct of this disclosure is exposed to one or more candidate drugs, and changes in FRET signals are monitored to determine whether the candidate modulates the activities of the proteolytic domain and/or the ligand-ligand binding domains interaction.

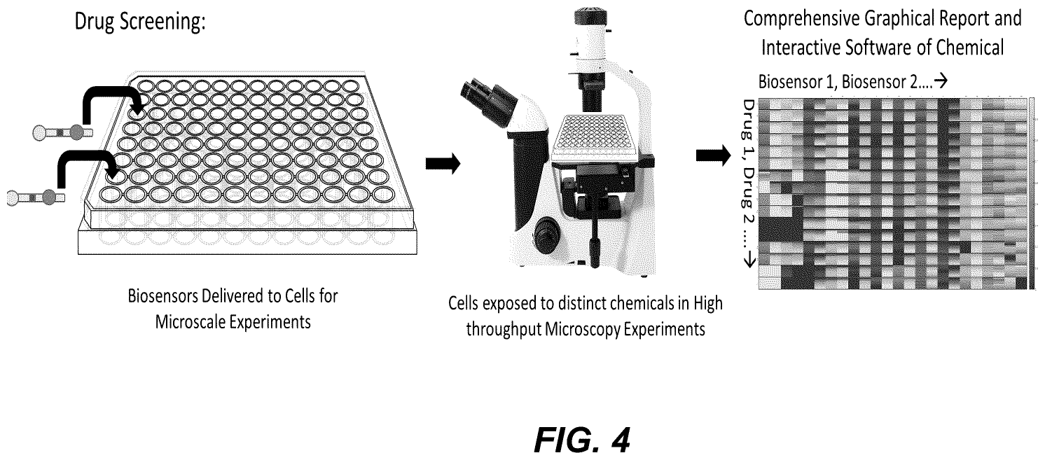

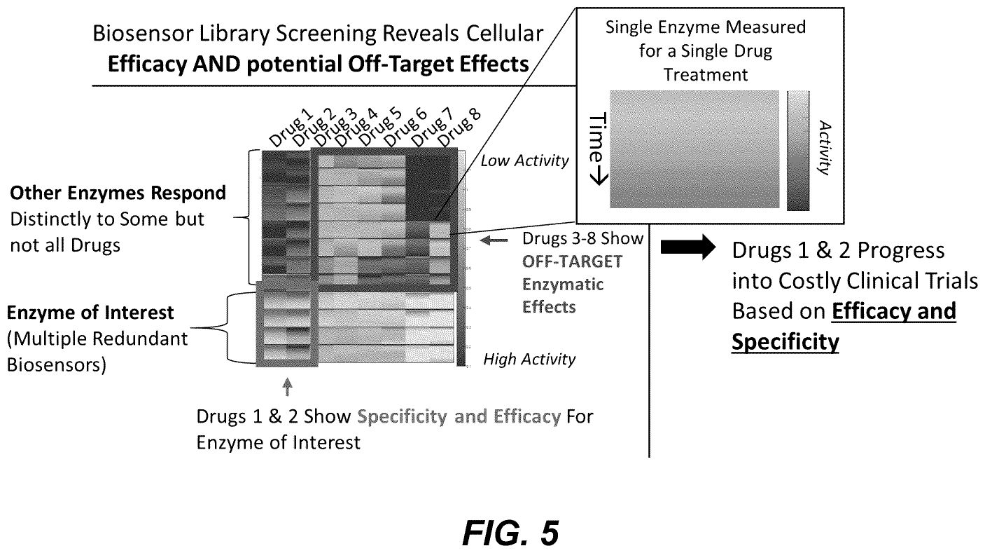

Referring to FIGS. 3 and 4, these methods of detecting drug candidate activity or screening putative inhibitors may be performed as a cell-based system in which a FRET biosensor construct of this disclosure is expressed inside a cell or on the surface of a cell, and the cell is then exposed to a sample comprising one or more drug candidates. Referring to FIG. 5, changes in FRET signals are then monitored as an indication of the presence, absence or specificity of the drug candidate activity.

In these methods, fluorescent detection and analysis can be carried out using, for example, a photon counting epifluorescent microscope system (containing the appropriate dichroic mirror and filters for monitoring fluorescent emission at the particular range), a photon counting photomultiplier system, or a fluorometer. Excitation to initiate energy transfer can be carried out with an argon ion laser, a high intensity mercury (Hg) arc lamp, a fiber optic light source, or other high intensity light source appropriately filtered for excitation in the desired range. It will be apparent to those skilled in the art that excitation/detection means can be augmented by the incorporation of photomultiplier means to enhance detection sensitivity. For example, the two-photon cross correlation method may be used to achieve the detection on a single-molecule scale (see e.g., Kohl et al., Proc. Natl. Acad. Sci., 99:12161, 2002).

Drug discovery projects, similar to these high throughput array assays, are often based on identifying a molecular target in an in vitro assay and performing a high-throughput screen of a library of chemical compounds. Identified drug candidates may be effective in modulating cell surface proteins or protein interactions. Such candidates may be modified through medicinal chemistry to optimize the chemical structure of the drug compound(s) for stability, delivery, and efficacy.

The agents to be screened by the high throughput methods of this disclosure can be any compound (e.g., an isolated compound), particularly any natural or synthetic chemical compound (such as a small molecule, including a combinatorial chemistry library of small molecules), organic compounds and molecules, inorganic compounds and molecules (e.g., heavy metal containing compounds), biological macromolecules (e.g., saccharides, lipids, peptides, proteins, polypeptides and nucleic acid molecules (e.g., those encoding a protein of interest)), inhibitory nucleic acid molecule (e.g., antisense or siRNA), and drugs (e.g., an FDA approved drug). In a particular embodiment, small molecules are screened using the methods of this disclosure.

### Kits

The present disclosure also provides kits for screening candidate drugs or drug libraries. The kits may comprise one or more FRET-based biosensor constructs of this disclosure, or polynucleotides encoding the biosensor(s). Instructions may be included which explain how to conduct the assay, how to detect fluorescence, and/or how to assess or correlate fluorescence to drug activity. Other optional reagents in the kit can include appropriate buffers for performing the assay. Kits may also comprise the isolated, recombinant, synthetic, or chimeric nucleic acid as provided herein, or vector, expression cassette, cosmid or plasmid as provided herein, or the cell, or cell line, or stable cell line as provided herein, and optionally further comprising instructions for practicing the method or array-based assay system provided herein.

### EXAMPLES

### Example 1

Preparing Biosensors that Report Different Biochemistry for Different Drug Responses

There are three ways side effects to a drug typically arise at the biochemical level: 1) A direct drug target regulates multiple physiological effects; 2) A drug has multiple direct targets, and 3) The metabolic breakdown product of a drug has distinct targets. In all three cases, a potential exists for pharmacological intervention, where the off-target mechanisms can be selectively inhibited, provided that these pathways can be detected and monitored in combinatorial drug screens for the discovery of such inhibitors. Live cell microscopy monitoring of biosensors within human cell lines is an inexpensive tool to perform such screening, as long as the collective ensemble of biosensors can widely survey all biochemical pathways. We have prepared and tested biosensor technology that utilizes FRET to convert binding events between biosensors and endogenous cellular molecules, such as endogenous proteins, into detectable changes in fluorescence. In some cases, these binding events result in proteolytic processing or other post translational modifications.

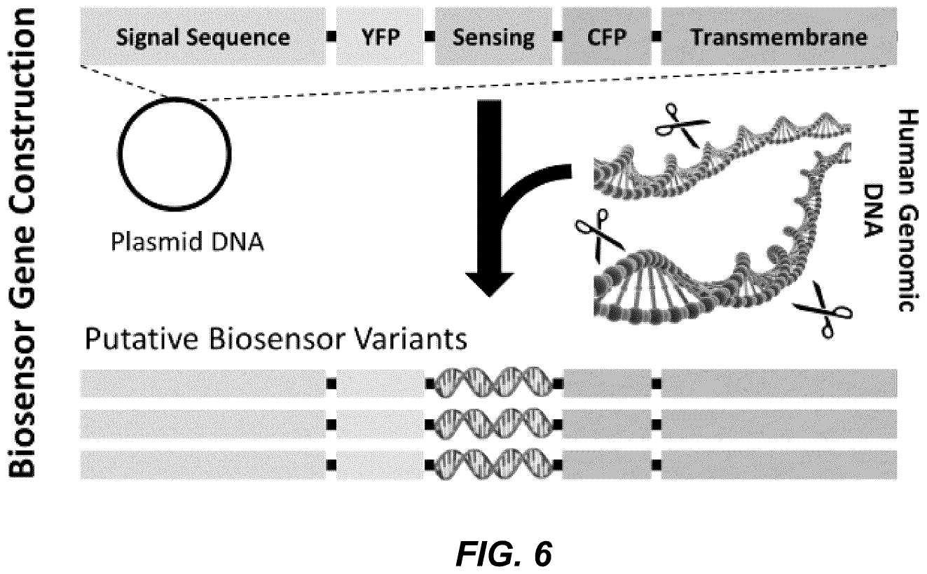

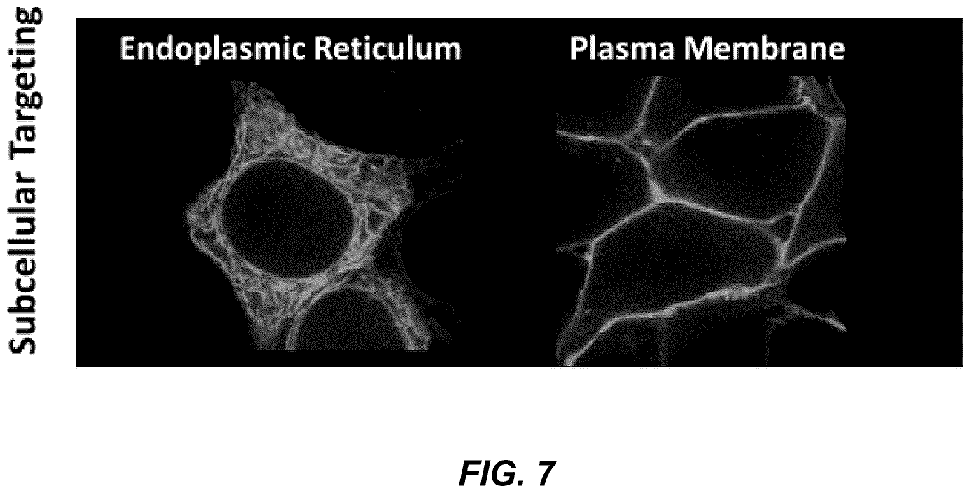

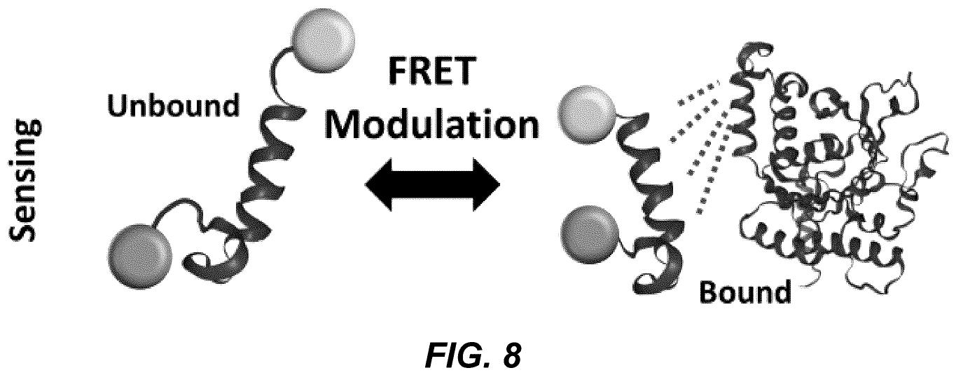

We have prepared biosensors based on the modular biosensor backbone design depicted in FIG. 6. This biosensor design was used to form a TNFα converting enzyme (TACE) biosensor to measure TACE activity in drug responses. This TACE biosensor functions by binding to the TACE enzyme, which enzymatically cleaves the biosensor into two protein products, measurable as changes in FRET signal in real time. Although our biosensors each consist of one continuous polypeptide made inside cells by cellular translation machinery, local regions within the polypeptide sequence perform distinct functions that enable diverse sensing abilities among the many possible variants in these biosensors. There are five ‘domains’ in the backbone design depicted in FIG. 6. The ‘signal sequence domain’ and the ‘transmembrane domain’ function in tandem to target a biosensor to either the plasma membrane or endoplasmic reticulum (ER) subcellular locations. The FRET capability is generated via the CFP (cyan fluorescent protein) domain and the YFP (yellow fluorescent protein) domain, which were chosen because of the efficient FRET that occurs when these two protein fluorophores are in close proximity (i.e., <10 nm). This FRET can be measured in biosensor-expressing cells by wide-field live cell fluorescent microscopy, where two images are taken of cells using either the CFP ex/CFP em fluorescent channel or the CFP ex/YFP em channel (see FIG. 7). These images are then processed with programs to quantify the changes in FRET signal throughout cells in time lapse microscopy videos. Changes in FRET reflect alteration in either the distance or orientation between the CFP and YFP fluorophores in a biosensor protein. This modulation of CFP/YFP distance and orientation arises when a biosensor protein binds to a cellular protein or is modified enzymatically (as is the case for the TACE biosensor) in such a way as to distort the polypeptide region (called the ‘sensing domain’) that connects the CFP and YFP domains. By substituting amino acid sequences into the ‘sensing domain’, we are able to influence the ability of a biosensor to bind to and be modified by endogenous proteins (depicted in FIG. 8). Thus, these biosensors report interactions with specific cellular proteins in the form of a dynamic FRET signal.

Feasibility experiments were conducted to probe the ability of these biosensors manufacturing and selection procedures to yield biosensors with diverse ability to report clusters of biochemical events within cells. 2,688 individual stable transgenic cell lines were generated, each expressing a unique variant of our prototype backbone in the human cell line, HEK293T. To accomplish this, we digested human genomic DNA with a strategic ensemble of endonucleases, such that the resulting products span approximately 500-1,000 bp. These double-stranded DNA fragments were inserted into the ‘sensing domain’ of our biosensor backbone (FIG. 6) using standard ligation techniques. The resulting plasmid products were transfected into human HEK293T cells using published techniques (Chapnick D A, Bunker E, Liu X. Sci Signal 2015,8:rs1; and Chapnick D A, Liu X., Mol Biol Cell 2014; 25:1586-93).

We determined the percentage of the 2,688 putative biosensors that possessed both robust sensing ability and specificity towards diverse drug responses, which allowed us to determine the scale required to generate a library of broad biosensor tools, each displaying unique reporter specificity to a pathway.

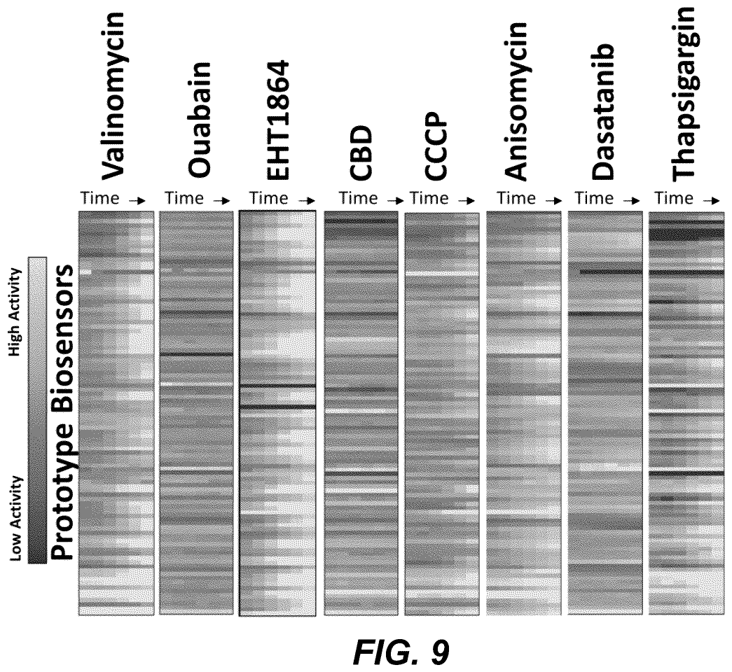

The selected biosensors exhibited specificity towards unique drug responses. 114 individual biosensors (4.2%) were identified from the test pool of 2,688 biosensors with robust sensing (dynamic drug modulation of signal/noise) through a selection method in which the biosensor-expressing cells are transferred to multiwell imaging dishes and screened in parallel live cell fluorescence microscopy experiments for the ability to report a dynamic change in FRET signal in response to parallel treatments of chemical modulators of biochemical pathways. A key outcome demonstrated in this experiment is that individual biosensors display unique drug response profiles across the eight-drug responses tested (FIG. 9). In other words, the resulting biosensors report different biochemistry for different drug responses.

### Example 2

### Detection of Companion Drug's Ability to Inhibit Pathways Within a Drug Response

An integrated data processing platform that combines state-of-the-art image analysis and machine learning techniques using the MATLAB (Mathworks) programming suite was used to construct and process multidimensional data. Biosensor activity (FRET signal) data has four dimensions that are tracked: ‘Biosensor Identity’, ‘Time’, ‘Single Drug Condition’, and ‘Combinatorial Drug Condition’. We used published methods to reduce the product images in microscopy experiments into activity profiles (Chapnick D A, Bunker E, Liu X., Sci Signal 2015,8:rs1 Chapnick D A, Liu X., Mol Biol Cell 2014; 25:1586-93; Chapnick D A, Jacobsen J, Liu X. PloS One 2013;8:e82444; Bennett CG, et al., Nucleic Acids Res 2016;44:3788-800).

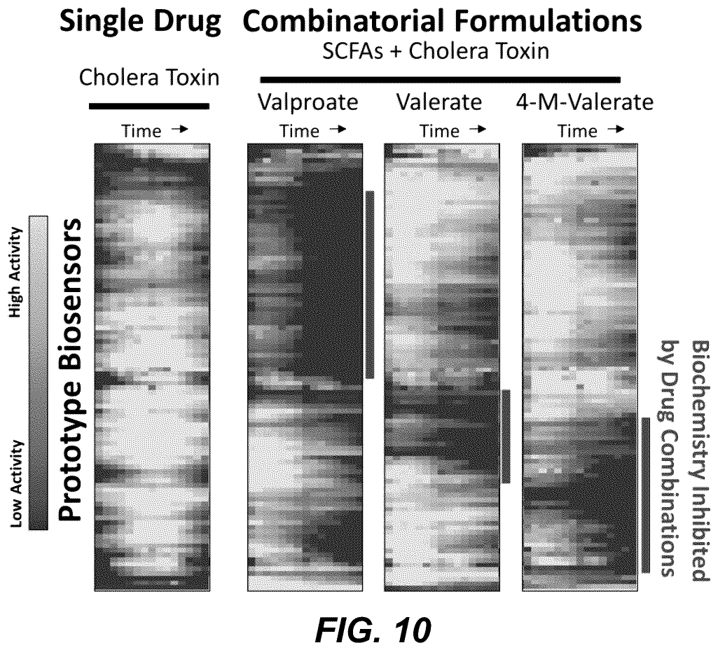

In these studies, four-dimensional data was used to profile a single drug response and combinatorial drug responses to construct concatenated two-dimensional arrays, followed by k-means clustering of biosensor identities along their time dependent activity profiles across all experimental conditions. For example, the drug response from Cholera Toxin-treated biosensor expressing cells display a relatively robust activity profile, where >80% of biosensors respond in some way to the single drug treatment (FIG. 10). When a combinatorial treatment of Cholera Toxin and one short chain fatty acid (SCFA) is performed, the Cholera Toxin biosensor response profile is uniquely distorted for each SCFA tested, despite that the SCFAs are chemical derivatives of each other. These unique profiles display how structurally similar compounds can have wide ranging ability to interfere with a given drug response. These data demonstrate the successful use of an automated scaled biosensor platform to screen drug response profiles in combinatorial drug libraries.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A method of evaluating drug activity comprising:
a) detecting whether the drug has activity in modulating ligand binding or proteolytic activity by contacting a biosensor with the drug, illuminating the sample, and detecting fluorescence resulting from a FRET energy transfer pair; and
c) determining the drug activity by comparing the fluorescence resulting from drug contact with the biosensor construct with a reference correlation of fluorescence and known or standard drug activity;
wherein the biosensor comprises from N-terminus to C-terminus:
1) a FRET acceptor or donor;
2) a proteolytic cleavage site;
3) a ligand;
4) a FRET acceptor or donor that completes a FRET energy transfer pair with the first position;
5) a ligand binding domain and/or a protease specific for the proteolytic cleavage site.

2. The method of claim 1, further comprising securing a plurality of the biosensors to a substrate surface at an addressable site forming an array of biosensors.

3. The method of claim 2, wherein the array comprises at least one biosensor secured to a living cell in cell culture.

4. The method of claim 3, wherein the array comprises at least one biosensor secured to the surface of each cell in the array.

5. The array of cells of claim 3, wherein the array comprises at least two biosensors comprising different proteolytic cleavage sites in the biosensor constructs.

6. The array of cells of claim 3, wherein the array comprises at least two biosensors comprising different ligands in the biosensor constructs.

7. The array of cells of claim 3, wherein the array comprises at least two biosensors comprising different ligand binding domain in the biosensor constructs.

8. The method of claim 1, further comprising evaluating drug specificity by contacting the drug with at least two biosensors, each comprising different proteolytic cleavage sites, and comparing the fluorescence resulting from drug contact with the different biosensor constructs to determine specificity of the drug in modulating the proteolytic cleavage of each biosensor.

9. The method of claim 1, further comprising evaluating drug specificity by contacting the drug with at least two biosensors, each comprising different ligands, and comparing the fluorescence resulting from drug contact with the different biosensor constructs to determine specificity of the drug in modulating the ligand binding domain interaction of each biosensor.

10. The method of claim 1, wherein the at least one biosensor comprises an array of biosensors comprising one or more different proteolytic cleavage sites and ligands or sensing domains.

11. The method of claim 10, wherein the array of biosensors is displayed on a membrane of living cells arranged in an array of cells in cell culture.

12. The method of claim 1, wherein the drug is contacted with the at least one biosensor within a composition comprising a library of candidate drugs.

13. A method of evaluating drug activity comprising:
a) detecting whether the drug has activity by contacting at least one biosensor with the drug, illuminating the sample, and detecting fluorescence resulting from the FRET energy transfer pair; and
c) determining the drug activity in the sample by comparing the fluorescence resulting from drug contact with the biosensor construct with a reference correlation of fluorescence and known or standard drug activity;
wherein the at least one biosensor comprises from N-terminus to C-terminus:
1) a FRET acceptor or donor;
2) a sensing domain comprising DNA; and
3) a FRET acceptor or donor that completes a FRET energy transfer pair with the first position.

14. The method of claim 13, further comprising securing a plurality of the biosensors to a substrate surface at an addressable site forming an array of biosensors.

15. The method of claim 14, wherein the array comprises at least one biosensor secured to a living cell in cell culture.

16. The method of claim 15, wherein the array comprises at least one biosensor on a surface of each cell in the array.

17. The array of cells of claim 15, wherein the array comprises at least two biosensors is comprising different sensing domains in the biosensor constructs.

18. The method of claim 13, further comprising evaluating drug specificity by contacting the drug with at least two biosensors, each comprising different sensing domains, and comparing the fluorescence resulting from drug contact with the different biosensor constructs to determine specificity of the drug in modulating the DNA binding of each biosensor.

19. The method of claim 13, wherein the at least one biosensor comprises an array of biosensors comprising one or more different sensing domains.

20. A method of evaluating drug activity from a library of biosensor constructs comprising:
generating at least two polynucleotides comprising different nucleic acid sequences;
inserting each of the at least two polynucleotides into separate biosensor constructs comprising, from N-terminus to C-terminus:
1) a FRET acceptor or donor;
2) a sensing domain comprising one of the at least two polynucleotides; and
3) a FRET acceptor or donor that completes a FRET energy transfer pair with the first position;
to form a library of biosensor constructs comprising sensing domains comprising different nucleic acid sequences.
